(19) United States
(12) Patent Application Publication
(10) Pub. No.: US 2024/0207075 A1
(43) Pub. Date: Jun. 27, 2024

(54) Absorbable Iron-Based Instrument

(71) Applicant: BIOTYX MEDICAL (SHENZHEN) CO., LTD., SHENZHEN, GUANGDONG (CN)

(72) Inventors: Dong Bian, Shenzhen (CN); Wenjiao Lin, Shenzhen (CN)

(73) Assignee: BIOTYX MEDICAL (SHENZHEN) CO., LTD., SHENZHEN, GUANGDONG (CN)

(21) Appl. No.: 17/802,448

(22) PCT Filed: May 27, 2020
(86) PCT No.: PCT/CN2020/092479

§ 371 Date: Aug. 25, 2022

(30) Foreign Application Priority Data
Dec. 30, 2019 (CN) CN201911399133.X

(51) Int. Cl. A61F 2/82; A61L 31/02; A61L 31/08; A61L 31/10; A61L 31/16

(52) U.S. Cl. CPC A61F 2/82; A61L 31/022; A61L 31/088; A61L 31/10; A61L 31/16; A61F 2250/0031; A61F 2250/0036; A61F 2250/0054; A61F 2250/0067

(57) ABSTRACT

Disclosed is an absorbable iron-based instrument (10). The absorbable iron-based instrument includes an iron-based matrix (100), a zinc-containing protective layer (200), a corrosion-promoting layer (300) and a drug controlled-release layer (400); the iron-based matrix (100) is provided with an outer wall (110), an inner wall (120) and a side wall (130); the zinc-containing protective layer (200) covers at least the outer wall (110) and the inner wall (120) of the iron-based matrix (100); the corrosion-promoting layer (300) fully covers the zinc-containing protective layer (200); the drug controlled-release layer (400) partially covers at least the corrosion-promoting layer (300); the corrosion-promoting layer (300) and the drug controlled-release layer (400) each contain degradable polymers; the weight-average molecular weight of the degradable polymers in the corrosion-promoting layer (300) is greater than the weight-average molecular weight of the degradable polymers in the drug controlled-release layer (400); and the thickness ratio of a portion of the zinc-containing protective layer (200) that is located at the inner wall (120) to a portion of the corrosion-promoting layer (300) that is located at the inner wall (120) is greater than the thickness ratio of a portion of the zinc-containing protective layer (200) that is located at the outer wall (110) to a portion of the corrosion-promoting layer (300) that is located at the outer wall (110). The corrosion behavior of the absorbable iron-based instrument (10) meets the requirements of clinical use, and the occurrence of adverse histological reactions is rare or avoided.

### TECHNICAL FIELD

The present invention relates to the field of medical instruments, in particular to an absorbable iron-based instrument.

### BACKGROUND ART

This section provides only background information related to the present invention, which is not necessarily the existing art.

At present, percutaneous coronary intervention (PCI) technology has become the main therapy for cardiovascular stenosis. Since the first procedure performed by Andreas Gruentzig in 1977, the PCI technology has continued to advance. Endovascular stents have evolved from a bare metal stent (BMS) to a drug-eluting stent (DES), and then to a biological resorbable stent (BRS).

The BRSs are mainly divided into two categories according to their materials. One category is degradable polymer stents, and the other category is corrodible metal stents. Polylactic acid is the most widely studied degradable polymer stent material. It has good biocompatibility, and its degradation products can be phagocytosed by cells and eventually metabolized into lactic acid, carbon dioxide and water. These metabolites can be completely absorbed by a human body. However, the mechanical properties of degradable polymers such as polylactic acid are significantly lower than those of metal materials. In order to ensure that a stent has a sufficient radial supporting force, a polymer stent needs to use more materials, which inevitably requires the polymer stent to have a larger bar width and wall thickness. A large sectional size of a stent bar will obviously affect the local hemodynamics and increase the risk of thrombosis. Therefore, the application of polymer stents is limited. A corrodible metal stent has excellent mechanical properties compared to a polymer stent. According to the material division, there are three main categories: a magnesium-based stent, a zinc-based stent and an iron-based stent. The mechanical properties of an iron-based stent are significantly better than those of a magnesium-based stent and a zinc-based stent, and are comparable to those of a traditional BMS and DES.

Existing studies have found that a bare iron-based stent is not corroded or is corroded slowly in animal bodies for a long time; and a full corrosion period may exceed 5 years, which cannot reflect the advantages of a BRS. After a degradable polymer coating is introduced to a surface of the stent, an acidic environment generated by polymer degradation can promote the corrosion of iron, which can regulate the degradation behavior of the iron stent and shorten the total degradation cycle. However, immediately after the stent is implanted, the degradable polymer coating begins to swell and be hydrolyzed, and an iron-based matrix starts to be corroded, which may cause the stent to lose an effective support prematurely before the completion of vascular remodeling, and subsequently cause restenosis. In order to ensure that the iron-based matrix does not start to be corroded within a certain period of time (3-6 months) after implantation, so as to provide a sufficient mechanical support for vascular repair, a zinc-containing protective layer is also introduced between the iron-based matrix and the degradable polymer layer. In addition, in order to make the degradation cycle of a degradable polymer and the consumption rate of the zinc-containing protective layer match the corrosion cycle of the iron-based matrix, so as to ensure that the iron-based matrix can provide a sufficient radial support during a repair period and that the iron-based matrix can be quickly corroded at the end of the repair period, it is crucial to select appropriate degradable polymers. Parameters such as the types and molecular weights of degradable polymers are key parameters affecting the consumption rate of a zinc-containing protector.

In order to slow down adverse tissue reactions and obtain better clinical effects, in the existing absorbable stent, an anti-proliferative drug, an anti-thrombotic drug and other drugs can also be introduced into the degradable polymer coating. A degradable polymer is used as a drug carrier. When the degradable polymer used as a drug carrier has an extremely large molecular weight, drugs are easily aggregated, resulting in poor drug dispersion, which will lead to serious drug release after implantation. Furthermore, the long-term drug release effect is poor; that is, the effective utilization rate of drugs is low, and the drug release is unreasonable; and it is hard to achieve an ideal treatment effect.

Therefore, how to synergize a zinc-containing protective layer, a degradable polymer layer, and a drug carrier to allow the corrosion behavior of an iron-based stent to meet clinical requirements, while slowing down the adverse histological reaction, remains to be solved.

### SUMMARY OF THE INVENTION

Based on this, it is necessary to provide an absorbable iron-based instrument, the corrosion behavior of which meets the requirements of clinical use, and which minimizes or avoids the occurrence of adverse histological reactions.

An absorbable iron-based instrument includes an iron-based matrix, a zinc-containing protective layer, a corrosion-promoting layer and a drug controlled-release layer; the iron-based matrix is provided with an outer wall, an inner wall and a side wall; the zinc-containing protective layer covers at least the outer wall and the inner wall of the iron-based matrix; the corrosion-promoting layer fully covers the zinc-containing protective layer; the drug controlled-release layer partially covers at least the corrosion-promoting layer; the corrosion-promoting layer and the drug controlled-release layer each contain degradable polymers; the weight-average molecular weight of the degradable polymer in the corrosion-promoting layer is greater than the weight-average molecular weight of the degradable polymer in the drug controlled-release layer; and the thickness ratio of a portion of the zinc-containing protective layer that is located at the inner wall to a portion of the corrosion-promoting layer that is located at the inner wall is greater than the thickness ratio of a portion of the zinc-containing protective layer that is located at the outer wall to a portion of the corrosion-promoting layer that is located at the outer wall.

In one embodiment, the weight-average molecular weight of the degradable polymer in the corrosion-promoting layer is at least twice as large as the weight-average molecular weight of the degradable polymer in the drug controlled-release layer.

In one embodiment, the weight-average molecular weight of the degradable polymer in the corrosion-promoting layer is 100-1000 kDa, and the weight-average molecular weight of the degradable polymer in the drug controlled-release layer is 8-50 kDa.

In one embodiment, the thickness ratio of the portion of the zinc-containing protective layer that is located at the inner wall to the portion of the corrosion-promoting layer that is located at the inner wall is 0.05-0.7, and the thickness ratio of the portion of the zinc-containing protective layer that is located at the outer wall to the portion of the corrosion-promoting layer that is located at the outer wall is 0.03-0.5.

In one embodiment, a thickness of the portion of the zinc-containing protective layer that is located at the outer wall is 0.5-2.0 microns, and a thickness of the portion of the zinc-containing protective layer that is located at the inner wall is 0.5-2.0 microns.

In one embodiment, the portion of the corrosion-promoting layer that is located at the outer wall has a thickness range from 4 to 15 microns, and the portion of the corrosion-promoting layer that is located at the inner wall has a thickness range from 3 to 10 microns.

In one embodiment, a thickness of the drug controlled-release layer is less than or equal to 5 microns.

In one embodiment, the material of the zinc-containing protective layer is pure zinc or a zinc alloy, and grain sizes of the pure zinc and the zinc alloy are both submicron.

In one embodiment, the zinc-containing protective layer covers the outer wall, the inner wall and the side wall of the iron-based matrix; the corrosion-promoting layer covers the entire surface of the zinc-containing protective layer; and the drug controlled-release layer covers at least the portion of the corrosion-promoting layer that is located at the outer wall.

In one embodiment, there is at least one corrosion-promoting layer; and when there is a plurality of corrosion-promoting layers, the degradable polymers in the different corrosion-promoting layers have different molecular weights and/or are of different types.

The above absorbable iron-based instrument uses the zinc-containing protective layer and the corrosion-promoting layer to jointly regulate the corrosion behavior of the iron-based matrix. In the early stage after implantation, the zinc-containing protective layer protects the iron-based matrix and delays the time point when the iron-based matrix starts to be corroded. A degradable polymer with a larger molecular weight is used to form the corrosion-promoting layer since it is degraded slowly. On the one hand, the corrosion-promoting layer can delay the consumption of the zinc-containing protective layer, thus further protecting the iron-based matrix in the early stage. On the other hand, the corrosion-promoting layer releases an acidic substance in the later stage of implantation to accelerate the corrosion of the iron-based matrix, so that the corrosion behavior of the instrument meets the requirements of clinical use.

Moreover, a degradable polymer with a smaller molecular weight is used as a drug carrier, which is beneficial to improve the effective utilization rate of drugs. Meanwhile, because the thickness ratio of the portion of the zinc-containing protective layer that is located at the inner wall to the portion of the corrosion-promoting layer that is located at the inner wall is greater than the thickness ratio of the portion of the zinc-containing protective layer that is located at the outer wall of the iron-based matrix to the portion of the corrosion-promoting layer that is located at the outer wall, the following phenomenon can be avoided: quick release of zinc ions in a short period of time that significantly increases thrombosis before endothelialization of the absorbable iron-based instrument after implantation, thereby reducing the risk of thrombosis. Therefore, it is beneficial to slow down or avoid adverse tissue reactions.

### BRIEF DESCRIPTION OF THE DRAWINGS

FIG. 1 is a schematic diagram of a section of a supporting rod of an absorbable iron-based instrument according to one embodiment;

FIG. 2 is a scanning electron microscope (SEM) diagram of iron-based stents of Embodiment 1 and Comparative Example 2;

FIG. 3 shows drug release curves of iron-based stents in Embodiments 1-11 and Comparative Examples 1-5 in a simulated body fluid; and

FIG. 4 shows corrosion behavior curves of iron-based stents in Embodiments 1-11 and Comparative Examples 1-5 in a simulated body fluid.

### DETAILED DESCRIPTION OF THE INVENTION

In order to make the foregoing objectives, features and advantages of the present invention more obvious and understandable, the specific implementation modes of the present invention are described in detail with reference to the accompanying drawings. Many specific details are described in the following descriptions to facilitate full understanding of the present invention. However, the present invention can be implemented in a variety of other ways than those described herein, and those skilled in the art can make similar improvements without departing from the connotation of the present invention. Therefore, the present invention is not limited by specific implementations disclosed below.

Unless otherwise defined, all technical and scientific terms used herein are the same as meanings of general understandings of those skilled in the art of the present invention. The terms used in the description of the present invention herein are merely to describe the specific embodiments, not intended to limit the present invention.

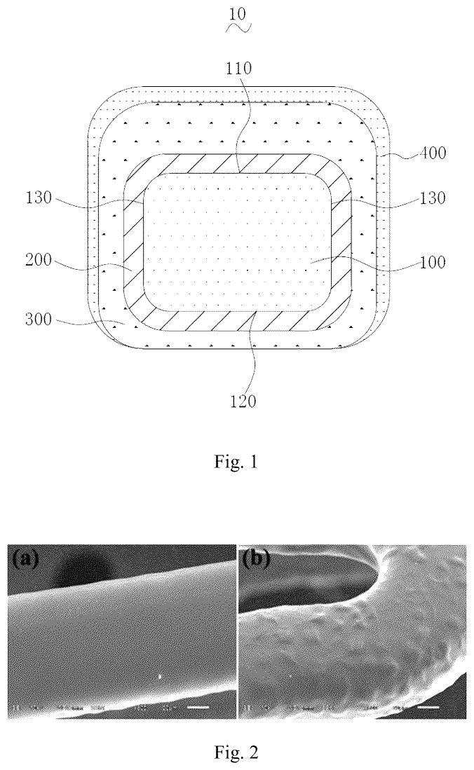

Referring to FIG. 1, an absorbable iron-based instrument 10 of one embodiment includes an iron-based matrix 100. The iron-based matrix 100 has a hollow lumen structure. The iron-based matrix 100 is provided with an outer wall 110, an inner wall 120 and a side wall 130.

In one embodiment, a material of the iron-based matrix 100 is pure iron or an iron alloy. The purity of the iron is not less than 99.9 wt %. An alloying element in the iron alloy is selected from at least one of carbon, nitrogen, phosphorus, silicon, sulfur, boron, cobalt, tungsten, manganese, tin, magnesium, zinc, zirconium, calcium, titanium, copper, gold, silver, platinum and palladium. In one implementation, a material of the iron-based matrix 100 is nitrided iron or carburized iron. The mechanical properties of the pure iron material are improved by nitriding or carburizing.

In another implementation, the material of the iron-based matrix 100 is not limited to the materials listed above. Any iron-based material which uses iron as a main component and can meet the requirements of early support and later quick corrosion of the iron-based matrix 100 can be used.

The absorbable iron-based instrument 10 also includes a zinc-containing protective layer 200, a corrosion-promoting layer 300 and a drug controlled-release layer 400 which are disposed on the iron-based matrix 100.

In one implementation, the zinc-containing protective layer 200 covers the outer wall 110, the inner wall 120 and the side wall 130 of the iron-based matrix 100. In one implementation, the zinc-containing protective layer 200 only covers the outer wall 110 and the inner wall 120 of the iron-based matrix 100. The material of the zinc-containing protective layer 200 is pure zinc or a zinc alloy. The purity of pure zinc is not less than 99.9 wt %. An alloying element in the zinc alloy is selected from at least one of magnesium, lithium, calcium, strontium, manganese, iron, tin, germanium, copper, bismuth, silver, gallium and zirconium. It should be noted that the content of the above alloying element is not enough to cause toxicity to a human body.

In one implementation, grain sizes of both the pure zinc and the zinc alloy are sub-micron.

No matter whether the zinc-containing protective layer 200 is made of pure zinc or a zinc alloy, the zinc-containing protective layer 200 has good compactness, and can isolate the iron-based matrix 100 from the body fluid when the absorbable iron-based instrument 10 is implanted into a living body. In addition, the zinc-containing protective layer 200 is preferentially corroded, and the corrosion of the iron-based matrix 100 is delayed. Meanwhile, corrosion products of the zinc-containing protective layer 200 adhere to the outer wall 110, the inner wall 120 and the side wall 130 of the iron-based matrix 100 to form a passivation film, which further protects the iron-based matrix 100 and further delays the time point when the iron-based matrix 100 starts to be corroded. This is favorable for maintaining the structural integrity of the iron-based matrix 100 in the early stage of implantation, so as to provide a sufficient radial support for a blood vessel and assist in the repair and remodeling of the blood vessel.

In one implementation, thicknesses of portions of the zinc-containing protective layer 200 that are located at the outer wall 110 and the inner wall 120 are both 0.5-2.0 microns.

In one implementation, the zinc-containing protective layer 200 is a film layer with a uniform thickness. When the zinc-containing protective layer 200 also covers the side wall 130, thicknesses of portions of the zinc-containing protective layer 200 that are located at the outer wall 110, the inner wall 120 and the side wall 130 are equal.

A preparation method of the zinc-containing protective layer 200 includes, but is not limited to, electroplating, chemical plating, spray coating, dip coating, brush coating, vapor deposition, magnetron sputtering, mechanical embedding, and ion implantation. In one implementation, when the material of the zinc-containing protective layer 200 is a zinc alloy, the zinc-containing protective layer 200 made of a zinc alloy material may be formed in situ by forming a pure zinc layer on a surface of the iron-based matrix 100 and then performing alloying.

The corrosion-promoting layer 300 covers the zinc-containing protective layer 200, and the corrosion-promoting layer 300 completely covers a surface of the iron-based matrix 100.

The material of the corrosion-promoting layer 300 is a material that can generate an acidic product during degradation to form a partial low-pH environment to promote the corrosion of the iron-based matrix 100, so that after the repair of the blood vessel is completed, the corrosion-promoting layer 300 can accelerate the corrosion of the iron-based matrix 100.

In one implementation, a material of the corrosion-promoting layer 300 contains a degradable polymer. The degradable polymer in the corrosion-promoting layer 300 is selected from at least one of degradable polyester and degradable acid anhydride.

In another implementation, the degradable polyester is selected from at least one of polylactic acid, polyglycolic acid, polylactic glycolic acid, polycaprolactone, polyacrylate, polyhydroxyalkanoate, polysuccinate, polysalicylic anhydride, polytrimethylene carbonate and polyethylene glycol ester. The degradable polyanhydride is selected from at least one of poly-1,3-bis(p-carboxy phenoxy)propane-sebacic acid, polyerucic acid dimer-sebacic acid, and polyfumaric acid-sebacic acid.

In one implementation, the degradable polymer is a copolymer obtained by copolymerizing at least two of monomers that form the above-mentioned degradable polyester and monomers that form the above-mentioned degradable acid anhydride.

In order to make the corrosion-promoting layer 300 not accelerate the corrosion of the iron-based matrix 100 and the zinc-containing protective layer 200 in the early stage and to accelerate the corrosion of the iron-based matrix 100 in the later stage, the molecular weight of the degradable polymer in the corrosion-promoting layer 300 should not be too small since a small molecular weight causes the degradable polymer to be degraded too quickly, so that it is difficult to achieve the above effect. In one implementation, the weight-average molecular weight of the degradable polymer in the corrosion-promoting layer 300 is 100-1000 kDa. If the weight average molecular weight is less than 100 kDa, the degradation rate is extremely large. If the weight-average molecular weight is greater than 1000 kDa, the degradable polymer is poor in the film-forming property and is difficult to prepare.

In one implementation, the corrosion-promoting layer 300 is a coating with a non-uniform thickness. For example, a thickness of a portion of the corrosion-promoting layer 300 that is located at the outer wall 110 of the iron-based matrix 100 is 4-15 microns, and a thickness of a portion located at the inner wall 120 is 3-10 microns.

There is at least one corrosion-promoting layer 300. When there are a plurality of corrosion-promoting layers 300, the degradable polymers in different corrosion-promoting layers 300 have different molecular weights or are of different types. Alternatively, the molecular weights and types of degradable polymers in different corrosion-promoting layers 300 are both different.

It should be noted that when there is a single corrosion-promoting layer 300, the above-mentioned 4-15 microns and 3-10 microns respectively refer to the thicknesses of the corresponding portions of the single corrosion-promoting layer 300. When there are a plurality of corrosion-promoting layers 300, the above-mentioned 4-15 microns and 3-10 microns respectively refer to sums of the thicknesses of the corresponding portions of the multiple corrosion-promoting layers 300.

A thickness ratio (referred to as an inner wall thickness ratio) of the portion of the zinc-containing protective layer 200 that is located at the inner wall 120 to the portion of the corrosion-promoting layer 300 that is located at the inner wall 120 is greater than a thickness ratio (referred to as an outer wall thickness ratio) of the portion of the zinc-containing protective layer 200 that is located at the outer wall 110 to the portion of the corrosion-promoting layer 300 that is located at the outer wall 110.

In one implementation, the inner wall thickness ratio is 0.05 to 0.7, and the outer wall thickness ratio is 0.03 to 0.5. That is, the inner wall thickness ratio can take any value from 0.05 to 0.7, and the outer wall thickness ratio can take any value from 0.03 to 0.5, but the inner wall thickness ratio should be greater than the outer wall thickness ratio.

It should be noted that, when there are a plurality of corrosion-promoting layers 300, the thicknesses of the corrosion-promoting layers 300 involved in the above-mentioned inner wall thickness ratio and outer wall thickness ratio refer to the sums of the thicknesses of the multiple corrosion-promoting layers 300.

After implantation, the zinc-containing protective layer 200 and the corrosion-promoting layer 300 are corroded and degraded at the same time in a body fluid environment. Furthermore, there is an interaction between them. Only when the degradation rate of the corrosion-promoting layer 300 matches the corrosion rate of the zinc-containing protective layer 200, the zinc-containing protective layer 200 can be protected in the early stage, thus avoiding fast corrosion of the zinc-containing protective layer 200 to better protect the iron-based matrix 100. There can be enough degradation products of the corrosion-promoting layer 300 in the later stage to generate a low-pH environment, thereby accelerating the corrosion of the iron-based matrix 100. In addition, the zinc-containing protective layer 200 continuously releases zinc ions, and the corrosion-promoting layer 300 continuously releases small-molecule polymer fragments. The zinc ions and the small-molecule polymer fragments are matched with each other, so that the corrosion products and the degradation products are released in safe doses, which avoids the phenomenon of a thrombus caused by hemolysis due to an extremely high concentration of zinc ions accumulated in local blood since the zinc-containing protective layer 200 is corroded too fast, and also avoids tissue inflammation caused by fast release of the degradation products of the corrosion-promoting layer 300.

The type and molecular weight of the degradable polymer in the corrosion-promoting layer 300 affect the degradation rate of the corrosion-promoting layer 300 itself, and the microstructure of the material of the zinc-containing protective layer 200 also affects the corrosion rate of the zinc-containing protective layer 200 itself. A suitable degradable polymer is selected to prepare the corrosion-promoting layer 300, and a suitable microstructure of the material of the zinc-containing retaining layer 200 is selected; and the thicknesses of the corrosion-promoting layer 300 and the zinc-containing retaining layer 200 are matched with each other, so that the iron-based matrix 100 can meet a mechanical support requirement in the early stage and be quickly corroded in the later stage on the premise that the use amounts of the corrosion-promoting layer 300 and the zinc-containing retaining layer 200 are as small as possible. In addition, when their use amounts are small, the zinc-containing protective layer 200 releases fewer zinc ions, and the corrosion-promoting layer 300 releases fewer degradation products, which is beneficial to reduce the safety problems caused by the release of the zinc ions and the degradation products, such as potential thrombosis risk and adverse histological reactions.

When the grain size of the pure zinc or zinc alloy in the zinc-containing protective layer 200 is submicron, the material of the corrosion-promoting layer 300 is the above-mentioned degradable polymer; and both the zinc-containing protective layer 200 and the corrosion-promoting layer 300 are arranged on the iron-based matrix 100 according to the above-mentioned inner wall thickness ratio and outer wall thickness ratio, which is beneficial to match their corrosion rates and degradation rates, and has had a desired effect on the corrosion behavior of the iron-based matrix 100.

In one implementation, when the thickness of the zinc-containing protective layer 200 is in the range of 0.5-2.0 microns, the molecular weight of the degradable polymer in the corrosion-promoting layer 300 is in the range of 100-1000 kDa; and when the thickness of the corrosion-promoting layer 300 is in the range of 3-15 microns, the use amounts and the releases can be controlled at a safety level on the premise of ensuring that the iron-based matrix 100 maintains enough mechanical properties within 3-6 months after implantation.

Through the synergistic effect of the zinc-containing protective layer 200 and the corrosion-promoting layer 300, after endothelium overlays the inner wall 120 of the iron-based matrix 100 to form an endothelial membrane layer, the iron-based matrix 100 starts to be corroded when a blood vessel can maintain its own shape. After the iron-based matrix 100 starts to be corroded, corrosion products of iron are relatively loose, and volume expansion will occur. If the volume-expanded portion is concentrated on a side close to the endothelial membrane layer, since the endothelial membrane layer is thin, the swelling corrosion products may stimulate the endothelial membrane film and cause hyperplasia, resulting in loss of a certain lumen.

Since the inner wall thickness ratio is greater than the outer wall thickness ratio, the portion of the zinc-containing protective layer 200 that is located at the outer wall 110 is consumed faster, and the iron-based matrix 100 starts to be corroded from the outer wall 110 first. Corrosion products develop and extend to a side away from the endothelial membrane layer, so as to reduce stimulation to the endothelial membrane layer. The corrosion products generated by the corrosion of the iron-based matrix 100 need to be phagocytosed by macrophages, and then transformed into hemosiderin which migrates to the vascular adventitia. Corrosion is initiated from the outer wall 110, so that a distance between the corrosion products of iron itself and the vascular adventitia is short; that is, the migration distance of metabolism of the corrosion products is short, which is beneficial to the metabolism and absorption of the corrosion products, thereby shortening the absorption cycle of the iron-based instrument 10.

The drug controlled-release layer 400 covers at least a partial region of the corrosion-promoting layer 300. In one embodiment, the drug controlled-release layer 400 at least covers a region of the corrosion-promoting layer 300 that is located at the outer wall 110.

The drug controlled-release layer 400 contains a degradable polymer and an active substance. The active substance is dispersed in the degradable polymer, and the degradable polymer is used as a carrier of the active substance. Under the same mass, a degradable polymer with a higher molecular weight has fewer end groups and fewer sites that can interact with the active substance, so the degradable polymer is directly used for carrying drugs. After implantation, the active substance is quickly released, but the active substance is slightly released in the later stage, making it difficult to achieve an effective therapeutic effect. Therefore, the molecular weight of the degradable polymer in the drug controlled-release layer 400 should not be too large.

A suitable degradable polymer is used as an active substance carrier to achieve a good drug controlled-release effect. However, in the release process of the active substance, with the degradation of the degradable polymer, the degradation products of the degradable polymer may affect the environment of the iron-based matrix 100, such as the pH value, thereby affecting the corrosion behaviors of the iron-based matrix 100 and the zinc-containing protective layer 200. Therefore, a suitable degradable polymer should be used as a carrier for the active substance by taking into account the drug controlled-release effect and avoiding the influence on the corrosion of the iron-based matrix 100.

In one embodiment, the weight-average molecular weight of the degradable polymer in the drug controlled-release layer 400 is 8-50 kDa. The degradable polymer within the weight average molecular weight range is used to carry drugs, so that an excellent drug release curve can be obtained, and at the same time, the corrosion of the iron-based matrix 100 and the corrosion of the zinc-containing protective layer 200 will not be significantly adversely affected.

In one embodiment, the degradable polymer in the drug controlled-release layer 400 is selected from at least one of degradable polyester and degradable acid anhydride.

In one implementation, the degradable polyester is selected from at least one of polylactic acid, polyglycolic acid, polylactic glycolic acid, polycaprolactone, polyacrylate, polyhydroxyalkanoate, polysuccinate, polysalicylic anhydride, polytrimethylene carbonate and polyethylene glycol ester. The degradable polyanhydride is selected from at least one of poly-1,3-bis(p-carboxy phenoxy)propane-sebacic acid, polyerucic acid dimer-sebacic acid, and polyfumaric acid-sebacic acid.

In another implementation, the degradable polymer is a copolymer obtained by copolymerizing at least two of monomers that form the above-mentioned degradable polyester and monomers that form the above-mentioned degradable acid anhydride.

A preparation method for the corrosion-promoting layer 300 and the drug controlled-release layer 400 includes, but is not limited to, spray coating, dip coating, brush coating and electrostatic spinning.

It should be noted that the types of the degradable polymer in the corrosion-promoting layer 300 and the degradable polymer in the drug controlled-release layer 400 may be the same or different.

In one implementation, the weight-average molecular weight of the degradable polymer in the corrosion-promoting layer 300 is at least twice as large as the weight-average molecular weight of the degradable polymer in the drug controlled-release layer 400.

In one embodiment, the active substance in the drug controlled-release layer 400 is an anti-proliferative drug, an anti-thrombotic drug, an anti-inflammatory drug, or an endothelialization-promoting substance. In one implementation, the anti-proliferative drug is at least one of paclitaxel, a paclitaxel derivative, rapamycin, and a rapamycin derivative. The anti-inflammatory drug is dexamethasone or the like. The endothelialization-promoting substance is at least one of a vascular endothelial growth factor, a fibroblast growth factor and a granulocyte colony stimulating factor. The anti-thrombotic drug is at least one of an anticoagulant drug, an antiplatelet drug and a thrombolytic drug.

In one implementation, the thickness of the drug controlled-release layer 400 is less than or equal to 5 microns.

In one embodiment, in either case that the drug controlled-release layer 400 only covers the portion of the corrosion-promoting layer 300 that is located at the outer wall 110 or completely covers the corrosion-promoting layer 300, a surface density of the active substance on the iron-based matrix 100 is 1.4 μg/mm².

The absorbable iron-based instrument 10 uses the zinc-containing protective layer 200 and the corrosion-promoting layer 300 to jointly regulate the corrosion behavior of the iron-based matrix 100. In the early stage after implantation, the zinc-containing protective layer 200 protects the iron-based matrix 100 so that the iron-based matrix 100 is not corroded, or is corroded relatively slowly. Meanwhile, the corrosion-promoting layer 300 is formed by using a degradable polymer with a larger molecular weight. The degradable polymer with the larger molecular weight is degraded slowly. On the one hand, the corrosion-promoting layer 300 can delay the consumption of the zinc-containing protective layer 200, thus further protecting the iron-based matrix 100 in the early stage. On the other hand, the corrosion-promoting layer 300 releases an acidic substance in the later stage of implantation to accelerate the corrosion of the iron-based matrix 100, so that the corrosion behavior of the absorbable iron-based instrument 10 meets the requirements of clinical use.

Moreover, a degradable polymer with a smaller molecular weight is used as a drug carrier, which is beneficial to improve the effective utilization rate of drugs. Meanwhile, the thickness ratio of the portion of the zinc-containing protective layer 200 that is located at the inner wall 120 to the portion of the corrosion-promoting layer 300 that is located at the inner wall 120 is greater than the thickness ratio of the portion of the zinc-containing protective layer 200 that is located at the outer wall of the iron-based matrix 100 to the portion of the corrosion-promoting layer 300 that is located at the outer wall 110, so that the following phenomenon can be avoided: the quick release of the zinc ions in a short period of time significantly increasing the thrombosis before endothelialization of the absorbable iron-based instrument 10 after implantation, thereby reducing the risk of thrombosis. Meanwhile, tissue inflammation generated by concentrated release of a large number of degradation products due to fast degradation of the corrosion-promoting layer 300 is avoided. Therefore, this is beneficial to slow down or avoid adverse tissue reactions.

The zinc-containing protective body layer 200, the corrosion-promoting layer 300 formed by the degradable polymer with the larger molecular weight and the drug controlled-release layer 400 formed by the degradable polymer with the smaller molecular weight are skillfully combined with a reasonable amount and a reasonable spatial layout to form a composite coating. The composite coating can make the absorbable iron-based instrument 10 adapt to tissue repair in terms of the mechanical properties, the degradation characteristic and the drug release, and can promote the repair of a lesion part and quickly restore its normal physiological functions. Compared with the traditional absorbable iron-based instrument, the absorbable iron-based instrument 10 has a higher endothelialization rate, lower thrombosis risk, more suitable corrosion and degradation behaviors, a shorter full absorption cycle, and better safety and efficacy.

In the absorbable iron-based instrument 10, the iron-based matrix 100 provides the basic structure of the entire instrument and provides a mechanical support required by the instrument. The synergistic effect of the zinc-containing protective layer 200, the corrosion-promoting layer 300 and the drug controlled-release layer 400 on the iron-based matrix 100 ensures that the iron-based matrix 100 is not corroded in the early stage (3-6 months) after implantation, can continuously release the active substance during the critical period of vascular repair (0˜90 days), and avoids an extremely high concentration of corrosion products and degradation products to prevent excessive tissue proliferation, restenosis and other adverse tissue reactions.

The entire system is skillfully combined into a whole to ensure the effectiveness and safety of the instrument.

The above absorbable iron-based instrument is further described below through specific embodiments.

The following embodiments use the following detection methods.

### 1. Detection Method for the Thicknesses of the Zinc-Containing Protective Layer, the Corrosion-Promoting Layer and the Drug Controlled-Release Layer:

### 1) Thicknesses of the Corrosion-Promoting Layer and the Drug Controlled-Release Layer

A SensofarQ-six vascular stent detector is used to detect the thicknesses of the corrosion-promoting layer and the drug controlled-release layer, and the test principle is optical coherence imaging. After the corrosion-promoting layer is prepared on the iron-based matrix, the thickness of the corrosion-promoting layer is detected. After the drug controlled-release layer is prepared, a total thickness of the polymer coating on the iron-based matrix is detected, and the difference between the two thicknesses is the thickness of the drug controlled-release layer. Three positions are uniformly selected in a longitudinal direction of each stent for thickness testing. Four stent bars uniformly distributed on a circumference are randomly selected at each position to test the thicknesses of the polymer coatings at the inner and outer walls. An average value of the 12 pieces of measured data is taken as an average thickness.

### 2) Thickness of the Zinc-Containing Protective Layer

Under an SEM, the stent is first subjected to metal (gold/platinum) spraying. For an extremely long stent, a segment that is required to be observed can be cut off for metal spraying and subsequent treatment. It is ensured that a surface of the stent or stent segment is completely covered. The purpose of metal spraying is to ensure that resin for embedding will not damage a surface of the zinc-containing protective layer during the subsequent embedding of a sample. The gold-sprayed sample is embedded in cold mounting resin, is then gradually ground from coarse to fine to expose a cross section of the stent, and is finally polished. The polished sample is pasted on an objective table of the SEM with a conductive adhesive, and is then observed and dimensioned after being subjected again to metal spraying. Three sections are selected as uniformly as possible along the longitudinal direction of the stent and are ground and polished. On the premise of ensuring that the zinc-containing protective layer on each surface of the stent is not damaged, four stent bars uniformly distributed on a circumference are randomly selected from each section. The thicknesses of the zinc-containing protective layer on inner and outer surfaces of the stent bars are measured. An average value of the 12 pieces of measured data is used as an average thickness of the zinc-containing protective layer.

### 2. Corrosion and Drug Release Test on the Stent

The stent is soaked in a simulated body fluid. The simulated body fluid is prepared from 10% (volume) of porcine plasma, 90% (volume) of PBS and 0.5 wt % of sodium azide. The PBS is prepared from 0.24 g of KH₂PO₄, 1.44 g of Na₂HPO₄, 8 g of NaCl and 0.2 g of KCl, and the pH is adjusted with HCl or NaOH to 7.4. Fresh simulated body fluid is replaced every 7 days.

After the stent is soaked in the simulated body fluid for a predetermined, time, the stent is taken out to observe changes of a surface of the stent; and a corrosion form of the stent is observed under a microscope and photographed. Later, the stent is put into an acetonitrile solution to ensure that the stent is completely submerged, and ultrasonic treatment is carried out for 20 minutes to dissolve a drug remaining on the surface of the stent. The acetonitrile solution with the dissolved drug is filtered through a 0.2-micron filter membrane; and a drug amount is tested on a high-performance liquid chromatograph. A drug release amount of the stent is obtained by subtracting an initial drug amount on the stent by a residual drug amount (the tested drug amount) on the soaked stent, and a drug release percentage is a ratio of the drug release amount to the initial drug amount. After the drug amount is tested, the stent is washed with an ethyl acetate solution to completely dissolve the polymer coating on the stent. The micro-CT examination is carried out on the above-mentioned cleaned stent, and a corrosion condition of the iron-based matrix is qualitatively analyzed. Next, the stent is ultrasonically cleaned in a tartaric acid solution to remove a corrosion product layer on the surface, and is weighed after it is dried. The mass loss rate of the iron-based matrix is defined as a percentage of a mass difference of the iron-based matrix before and after implantation to the mass of the iron-based matrix before implantation.

In the following embodiments, a stent of the specification of 30008 is used as a test sample for description. The stent of the specification of 30008 is defined as follows: Under the action of a nominal expansion pressure of 8 atm, the stent has a nominal diameter after expansion of 3.0 mm and a nominal length of 8.0 mm.

### Embodiment 1

A specific preparation method of an iron-based stent is as follows: A nitrided iron-based matrix of a specification of 30008 with a wall thickness of 50 microns and an inner diameter of 1.45 mm was galvanized using an electroplating method to obtain a zinc-containing protective layer that covered an entire surface of the iron-based matrix and had a uniform thickness (1 micron). The grain size of zinc in the zinc-containing protective layer was submicron. Later, an ethyl acetate solution of PDLLA with a weight-average molecular weight of 200 kDa was spray-coated, and after a solvent was dried, a corrosion-promoting layer that completely covered the zinc-containing protective layer was obtained. In the spray-coating process, one polytetrafluoroethylene (PTFE) mandrel was added inside the iron-based matrix to obtain the corrosion-promoting layer with different thicknesses at the inner and outer walls. A portion of the corrosion-promoting layer that was located at an outer wall of the iron-based matrix had a thickness of 10 microns, and a portion located at an inner wall had a thickness of 8 microns. An inner wall thickness ratio was 0.13, which was greater than an outer wall thickness ratio of 0.10. PDLLA, with a weight-average molecular weight of 8 kDa, was selected as a drug-loading polymer of the drug controlled-release layer; the ethyl acetate mixed solution of the PDLLA and sirolimus was spray-coated on a surface of the corrosion-promoting layer; in the spray-coating process, one PTFE mandrel was added inside the matrix, which completely blocked away the sprayed solution to prevent the solution from being deposited on the inner wall of the matrix; and after drying, the drug controlled-release layer that only covered the outer wall and a side wall of the matrix was obtained. A portion of the drug controlled-release layer that was located at the outer wall had a thickness of 2 microns, and the stent had a drug surface density of 1.4 μg/mm². The ratio of the molecular weight of the polymer in the corrosion-promoting layer to the molecular weight of the polymer in the drug controlled-release layer was 25.0.

The above stent was soaked in simulated body fluid; the whole stent was placed on a 37° C. constant-temperature air bath shaker; the simulated body fluid was replaced with fresh simulated body fluid every 7 days; after being soaked for 1, 7, 14, 28, and 60 days, the stent was taken out to test a drug release percentage and an iron-based matrix mass loss rate. The drug release percentages of the stent at various time points were 9.31%, 19.42%, 31.50%, 49.70% and 64.11% respectively. The mass loss rates of the stent at various time points were 0.62%, 0.77%, 6.95%, 12.29% and 43.67% respectively, and the corrosion of the iron-based matrix was initiated from an outer surface of the stent.

### Embodiment 2

A specific preparation method of an iron-based stent is as follows: A nitrided iron-based matrix of a specification of 30008 with a wall thickness of 50 microns and an inner diameter of 1.45 mm was galvanized using an electroplating method to obtain a zinc-containing protective layer that covered an entire surface of the iron-based matrix and had a uniform thickness (1 micron). The grain size of zinc in the zinc-containing protective layer was submicron. Later, an ethyl acetate solution of PDLLA with a weight-average molecular weight of 200 kDa was spray-coated, and after a solvent was dried, a corrosion-promoting layer that completely covered the zinc-containing protective layer was obtained. In the spray-coating process, one PTFE mandrel was added inside the iron-based matrix to obtain the corrosion-promoting layer with different thicknesses at the inner and outer walls. A portion of the corrosion-promoting layer that was located at an outer wall of the iron-based matrix had a thickness of 10 microns, and a portion located at an inner wall had a thickness of 8 microns. An inner wall thickness ratio was 0.13, which was greater than an outer wall thickness ratio of 0.10. PDLLA, with a weight-average molecular weight of 10 kDa, was selected as a drug-loading polymer of the drug controlled-release layer; the ethyl acetate mixed solution of the PDLLA and sirolimus was spray-coated on a surface of the corrosion-promoting layer; in the spray-coating process, one PTFE mandrel was added inside the matrix, which completely blocked away the sprayed solution to prevent the solution from being deposited on the inner wall of the matrix; and after drying, the drug controlled-release layer that only covered the outer wall and a side wall of the matrix was obtained. A portion of the drug controlled-release layer that was located at the outer wall had a thickness of 2 microns, and the stent had a drug surface density of 1.4 μg/mm². The ratio of the molecular weight of the polymer in the corrosion-promoting layer to the molecular weight of the polymer in the drug controlled-release layer was 20.0.

The above stent was soaked in simulated body fluid; the whole stent was placed on a 37° C. constant-temperature air bath shaker; the simulated body fluid was replaced with fresh simulated body fluid every 7 days; after being soaked for 1, 7, 14, 28, and 60 days, the stent was taken out to test a drug release percentage and an iron-based matrix mass loss rate. The drug release percentages of the stent at various time points were 10.44%, 19.55%, 20.96%, 32.81% and 49.07% respectively. The mass loss rates of the stent at various time points were 0.34%, 0.87%, 6.55%, 9.98% and 40.36% respectively, and the corrosion of the iron-based matrix was initiated from an outer surface of the stent.

### Embodiment 3

A specific preparation method of an iron-based stent is as follows: A nitrided iron-based matrix of a specification of 30008 with a wall thickness of 50 microns and an inner diameter of 1.45 mm was galvanized using an electroplating method to obtain a zinc-containing protective layer that covered an entire surface of the iron-based matrix and had a uniform thickness (1 micron). The grain size of zinc in the zinc-containing protective layer was submicron. Later, an ethyl acetate solution of PDLLA with a weight-average molecular weight of 200 kDa was spray-coated, and after a solvent was dried, a corrosion-promoting layer that completely covered the zinc-containing protective layer was obtained. In the spray-coating process, one PTFE mandrel was added inside the iron-based matrix to obtain the corrosion-promoting layer with different thicknesses at the inner and outer walls. A portion of the corrosion-promoting layer that was located at an outer wall of the iron-based matrix had a thickness of 10 microns, and a portion located at an inner wall had a thickness of 8 microns. An inner wall thickness ratio was 0.13, which was greater than an outer wall thickness ratio of 0.10. PDLLA, with a weight-average molecular weight of 30 kDa, was selected as a drug-loading polymer of the drug controlled-release layer; the ethyl acetate mixed solution of the PDLLA and sirolimus was spray-coated on a surface of the corrosion-promoting layer; in the spray-coating process, one PTFE mandrel was added inside the matrix, which completely blocked away the sprayed solution to prevent the solution from being deposited on the inner wall of the matrix; and after drying, the drug controlled-release layer that only covered the outer wall and a side wall of the matrix was obtained. A portion of the drug controlled-release layer that was located at the outer wall had a thickness of 2 microns, and the stent had a drug surface density of 1.4 μg/mm². The ratio of the molecular weight of the polymer in the corrosion-promoting layer to the molecular weight of the polymer in the drug controlled-release layer was 6.7.

The above stent was soaked in simulated body fluid; the whole stent was placed on a 37° C. constant-temperature air bath shaker; the simulated body fluid was replaced with fresh simulated body fluid every 7 days; after being soaked for 1, 7, 14, 28, and 60 days, the stent was taken out to test a drug release percentage and an iron-based matrix mass loss rate. The drug release percentages of the stent at various time points were 15.02%, 20.40%, 27.54%, 39.13% and 45.11% respectively. The mass loss rates of the stent at various time points were 0.54%, 0.65%, 5.21%, 8.99% and 40.45% respectively, and the corrosion of the iron-based matrix was initiated from an outer surface of the stent.

### Embodiment 4

A specific preparation method of an iron-based stent is as follows: A nitrided iron-based matrix of a specification of 30008 with a wall thickness of 50 microns and an inner diameter of 1.45 mm was galvanized using an electroplating method to obtain a zinc-containing protective layer that covered an entire surface of the iron-based matrix and had a uniform thickness (1 micron). The grain size of zinc in the zinc-containing protective layer was submicron. Later, an ethyl acetate solution of PDLLA with a weight-average molecular weight of 200 kDa was spray-coated, and after a solvent was dried, a corrosion-promoting layer that completely covered the zinc-containing protective layer was obtained. In the spray-coating process, one PTFE mandrel was added inside the iron-based matrix to obtain the corrosion-promoting layer with different thicknesses at the inner and outer walls. A portion of the corrosion-promoting layer that was located at an outer wall of the iron-based matrix had a thickness of 10 microns, and a portion located at an inner wall had a thickness of 8 microns. An inner wall thickness ratio was 0.13, which was greater than an outer wall thickness ratio of 0.10. PDLLA, with a weight-average molecular weight of 50 kDa, was selected as a drug-loading polymer of the drug controlled-release layer; the ethyl acetate mixed solution of the PDLLA and sirolimus was spray-coated on a surface of the corrosion-promoting layer; in the spray-coating process, one PTFE mandrel was added inside the matrix, which completely blocked away the sprayed solution to prevent the solution from being deposited on the inner wall of the matrix; and after drying, the drug controlled-release layer that only covered the outer wall and a side wall of the matrix was obtained. A portion of the drug controlled-release layer that was located at the outer wall had a thickness of 2 microns, and the stent had a drug surface density of 1.4 μg/mm². The ratio of the molecular weight of the polymer in the corrosion-promoting layer to the molecular weight of the polymer in the drug controlled-release layer was 4.0.

The above stent was soaked in simulated body fluid; the whole stent was placed on a 37° C. constant-temperature air bath shaker; the simulated body fluid was replaced with fresh simulated body fluid every 7 days; after being soaked for 1, 7, 14, 28, and 60 days, the stent was taken out to test a drug release percentage and an iron-based matrix mass loss rate. The drug release percentages of the stent at various time points were 21.10%, 30.30%, 38.66%, 51.20% and 57.90% respectively. The mass loss rates of the stent at various time points were 0.21%, 0.87%, 4.21%, 7.88% and 39.89% respectively, and the corrosion of the iron-based matrix was initiated from an outer surface of the stent.

The above stent was implanted into the iliac arteries of rabbits; the rabbits were killed after different raising times; and the implanted vessel segments were taken out for analysis. After 10 days of implantation of the stent, obvious overlaying of endothelial cells was observed; and after 28 days, the stent had undergone complete endothelialization. The drug release percentage after 1 day of implantation of the stent was 15.3%; the drug release percentage after 30 days was 45.7%; the drug release percentage after 60 days was 67.6%; the drug release percentage after 90 days was 84.9%; and the drug release percentage after 180 days was 96.4%. It was observed that the iron-based matrix started to be corroded (the mass loss rate was about 5%) after 120 days of implantation of the stent; the mass loss rate after 180 days of implantation was 25%; and the iron-based stent was fully corroded (the mass loss rate was greater than 95%) after 1.5 years of implantation. During the entire follow-up period, no thrombi were observed in the inner wall of the stent and the implanted vessel segments; and the lumen was clear, without restenosis.

### Embodiment 5

A specific preparation method of an iron-based stent is as follows: A nitrided iron-based matrix of a specification of 30008 with a wall thickness of 50 microns and an inner diameter of 1.45 mm was galvanized using an electroplating method to obtain a zinc-containing protective layer that covered an entire surface of the iron-based matrix and had a uniform thickness (1 micron). The grain size of zinc in the zinc-containing protective layer was submicron. Later, an ethyl acetate solution of PLLA with a weight-average molecular weight of 100 kDa was spray-coated, and after a solvent was dried, a corrosion-promoting layer that completely covered the zinc-containing protective layer was obtained. In the spray-coating process, one PTFE mandrel was added inside the iron-based matrix to obtain the corrosion-promoting layer with different thicknesses at the inner and outer walls. A portion of the corrosion-promoting layer that was located at an outer wall of the iron-based matrix had a thickness of 10 microns, and a portion located at an inner wall had a thickness of 8 microns. An inner wall thickness ratio was 0.13, which was greater than an outer wall thickness ratio of 0.10. PDLLA, with a weight-average molecular weight of 30 kDa, was selected as a drug-loading polymer of the drug controlled-release layer; the ethyl acetate mixed solution of the PDLLA and sirolimus was spray-coated on a surface of the corrosion-promoting layer; in the spray-coating process, one PTFE mandrel was added inside the matrix, which completely blocked away the sprayed solution to prevent the solution from being deposited on the inner wall of the matrix; and after drying, the drug controlled-release layer that only covered the outer wall and a side wall of the matrix was obtained. A portion of the drug controlled-release layer that was located at the outer wall had a thickness of 2 microns, and the stent had a drug surface density of 1.4 μg/mm². The ratio of the molecular weight of the polymer in the corrosion-promoting layer to the molecular weight of the polymer in the drug controlled-release layer was 3.3.

The above stent was soaked in simulated body fluid; the whole stent was placed on a 37° C. constant-temperature air bath shaker; the simulated body fluid was replaced with fresh simulated body fluid every 7 days; after being soaked for 1, 7, 14, 28, and 60 days, the stent was taken out to test a drug release percentage and an iron-based matrix mass loss rate. The drug release percentages of the stent at various time points were 14.70%, 19.71%, 27.62%, 41.20% and 46.31% respectively. The mass loss rates of the stent at various time points were 2.45%, 3.54%, 8.97%, 18.21% and 55.43% respectively, and the corrosion of the iron-based matrix was initiated from an outer surface of the stent.

### Embodiment 6

A specific preparation method of an iron-based stent is as follows: A nitrided iron-based matrix of a specification of 30008 with a wall thickness of 50 microns and an inner diameter of 1.45 mm was galvanized using an electroplating method to obtain a zinc-containing protective layer that covered an entire surface of the iron-based matrix and had a uniform thickness (1 micron). The grain size of zinc in the zinc-containing protective layer was submicron. Later, an ethyl acetate solution of PLLA with a weight-average molecular weight of 500 kDa was spray-coated, and after a solvent was dried, a corrosion-promoting layer that completely covered the zinc-containing protective layer was obtained. In the spray-coating process, one PTFE mandrel was added inside the iron-based matrix to obtain the corrosion-promoting layer with different thicknesses at the inner and outer walls. A portion of the corrosion-promoting layer that was located at an outer wall of the iron-based matrix had a thickness of 10 microns, and a portion located at an inner wall had a thickness of 8 microns. An inner wall thickness ratio was 0.13, which was greater than an outer wall thickness ratio of 0.10. PDLLA, with a weight-average molecular weight of 30 kDa, was selected as a drug-loading polymer of the drug controlled-release layer; the ethyl acetate mixed solution of the PDLLA and sirolimus was spray-coated on a surface of the corrosion-promoting layer; in the spray-coating process, one PTFE mandrel was added inside the matrix, which completely blocked away the sprayed solution to prevent the solution from being deposited on the inner wall of the matrix; and after drying, the drug controlled-release layer that only covered the outer wall and a side wall of the matrix was obtained. A portion of the drug controlled-release layer that was located at the outer wall had a thickness of 2 microns, and the stent had a drug surface density of 1.4 μg/mm². The ratio of the molecular weight of the polymer in the corrosion-promoting layer to the molecular weight of the polymer in the drug controlled-release layer was 16.7.

The above stent was soaked in simulated body fluid; the whole stent was placed on a 37° C. constant-temperature air bath shaker; the simulated body fluid was replaced with fresh simulated body fluid every 7 days; after being soaked for 1, 7, 14, 28, and 60 days, the stent was taken out to test a drug release percentage and an iron-based matrix mass loss rate. The drug release percentages of the stent at various time points were 14.5%, 19.8%, 26.5%, 38.9% and 42.3% respectively. The mass loss rates of the stent at various time points were 0.12%, 0.36%, 1.76%, 3.21% and 17.89% respectively, and the corrosion of the iron-based matrix was initiated from an outer surface of the stent.

### Embodiment 7

A specific preparation method of an iron-based stent is as follows: A nitrided iron-based matrix of a specification of 30008 with a wall thickness of 50 microns and an inner diameter of 1.45 mm was galvanized using an electroplating method to obtain a zinc-containing protective layer that covered an entire surface of the iron-based matrix and had a uniform thickness (1 micron). The grain size of zinc in the zinc-containing protective layer was submicron. Later, an ethyl acetate solution of PDLLA with a weight-average molecular weight of 1000 kDa was spray-coated, and after a solvent was dried, a corrosion-promoting layer that completely covered the zinc-containing protective layer was obtained. In the spray-coating process, one PTFE mandrel was added inside the iron-based matrix to obtain the corrosion-promoting layer with different thicknesses at the inner and outer walls. A portion of the corrosion-promoting layer that was located at an outer wall of the iron-based matrix had a thickness of 10 microns, and a portion located at an inner wall had a thickness of 8 microns. An inner wall thickness ratio was 0.13, which was greater than an outer wall thickness ratio of 0.10. PDLLA, with a weight-average molecular weight of 30 kDa, was selected as a drug-loading polymer of the drug controlled-release layer; the ethyl acetate mixed solution of the PDLLA and sirolimus was spray-coated on a surface of the corrosion-promoting layer; in the spray-coating process, one PTFE mandrel was added inside the matrix, which completely blocked away the sprayed solution to prevent the solution from being deposited on the inner wall of the matrix; and after drying, the drug controlled-release layer that only covered the outer wall and a side wall of the matrix was obtained. A portion of the drug controlled-release layer that was located at the outer wall had a thickness of 2 microns, and the stent had a drug surface density of 1.4 μg/mm². The ratio of the molecular weight of the polymer in the corrosion-promoting layer to the molecular weight of the polymer in the drug controlled-release layer was 33.3.

The above stent was soaked in simulated body fluid; the whole was placed on a 37° C. constant-temperature air bath shaker; the simulated body fluid was replaced with fresh simulated body fluid every 7 days; after being soaked for 1, 7, 14, 28, and 60 days, the stent was taken out to test a drug release percentage and an iron-based matrix mass loss rate. The drug release percentages of the stent at various time points were 14.3%, 21.2%, 28.9%, 41.1% and 47.1% respectively. The mass loss rates of the stent at various time points were 0.31%, 0.78%, 0.98%, 1.45% and 2.34% respectively, and the corrosion of the iron-based matrix was initiated from an outer surface of the stent.

### Embodiment 8

A specific preparation method of an iron-based stent is as follows: A nitrided iron-based matrix of a specification of 30008 with a wall thickness of 50 microns and an inner diameter of 1.45 mm was galvanized using a chemical plating method to obtain a zinc-containing protective layer that covered an entire surface of the iron-based matrix and had a uniform thickness (2 microns). The grain size of zinc in the zinc-containing protective layer was submicron. Later, an ethyl acetate solution of PDLLA with a weight-average molecular weight of 200 kDa was spray-coated, and after a solvent was dried, a corrosion-promoting layer that completely covered the zinc-containing protective layer was obtained. In the spray-coating process, one PTFE mandrel was added inside the iron-based matrix to obtain the corrosion-promoting layer with different thicknesses at the inner and outer walls. A portion of the corrosion-promoting layer that was located at an outer wall of the iron-based matrix had a thickness of 15 microns, and a portion located at an inner wall had a thickness of 10 microns. An inner wall thickness ratio was 0.20, which was greater than an outer wall thickness ratio of 0.13. PDLLA, with a weight-average molecular weight of 30 kDa, was selected as a drug-loading polymer of the drug controlled-release layer; the ethyl acetate mixed solution of the PDLLA and sirolimus was spray-coated on a surface of the corrosion-promoting layer; in the spray-coating process, one PTFE mandrel was added inside the matrix, which completely blocked away the sprayed solution to prevent the solution from being deposited on the inner wall of the matrix; and after drying, the drug controlled-release layer that only covered the outer wall and a side wall of the matrix was obtained. A portion of the drug controlled-release layer that was located at the outer wall had a thickness of 2 microns, and the stent had a drug surface density of 1.4 μg/mm². The ratio of the molecular weight of the polymer in the corrosion-promoting layer to the molecular weight of the polymer in the drug controlled-release layer was 6.7.

The above stent was soaked in simulated body fluid; the whole stent was placed on a 37° C. constant-temperature air bath shaker; the simulated body fluid was replaced with fresh simulated body fluid every 7 days; after being soaked for 1, 7, 14, 28, and 60 days, the stent was taken out to test a drug release percentage and an iron-based matrix mass loss rate. The drug release percentages of the stent at various time points were 17.0%, 21.4%, 28.9%, 41.3% and 47.6% respectively. The mass loss rates of the stent at various time points were 1.52%, 0.93%, 2.3%, 6.7% and 39.17% respectively, and the corrosion of the iron-based matrix was initiated from an outer surface of the stent.

### Embodiment 9

A specific preparation method of an iron-based stent is as follows: A nitrided iron-based matrix of a specification of 30008 with a wall thickness of 50 microns and an inner diameter of 1.45 mm was galvanized using a chemical plating method to obtain a zinc-containing protective layer that covered an entire surface of the iron-based matrix and had a uniform thickness (0.5 microns). The grain size of zinc in the zinc-containing protective layer was submicron. Later, an ethyl acetate solution of PDLLA with a weight-average molecular weight of 200 kDa was spray-coated, and after a solvent was dried, a corrosion-promoting layer that completely covered the zinc-containing protective layer was obtained. In the spray-coating process, one PTFE mandrel was added inside the iron-based matrix to obtain the corrosion-promoting layer with different thicknesses at the inner and outer walls. A portion of the corrosion-promoting layer that was located at an outer wall of the iron-based matrix had a thickness of 15 microns, and a portion located at an inner wall had a thickness of 10 microns. An inner wall thickness ratio was 0.05, which was greater than an outer wall thickness ratio of 0.03. PDLLA, with a weight-average molecular weight of 30 kDa, was selected as a drug-loading polymer of the drug controlled-release layer; the ethyl acetate mixed solution of the PDLLA and sirolimus was spray-coated on a surface of the corrosion-promoting layer; in the spray-coating process, one PTFE mandrel was added inside the matrix, which completely blocked away the sprayed solution to prevent the solution from being deposited on the inner wall of the matrix; and after drying, the drug controlled-release layer that only covered the outer wall and a side wall of the matrix was obtained. A portion of the drug controlled-release layer that was located at the outer wall had a thickness of 2 microns, and the stent had a drug surface density of 1.4 μg/mm². The ratio of the molecular weight of the polymer in the corrosion-promoting layer to the molecular weight of the polymer in the drug controlled-release layer was 6.7.

The above stent was soaked in simulated body fluid; the whole stent was placed on a 37° C. constant-temperature air bath shaker; the simulated body fluid was replaced with fresh simulated body fluid every 7 days; after being soaked for 1, 7, 14, 28, and 60 days, the stent was taken out to test a drug release percentage and an iron-based matrix mass loss rate. The drug release percentages of the stent at various time points were 16.5%, 23.4%, 29.7%, 41.2% and 47.3% respectively. The mass loss rates of the stent at various time points were 1.54%, 7.34%, 17.89%, 39.78% and 64.32% respectively, and the corrosion of the iron-based matrix was initiated from an outer surface of the stent.

### Embodiment 10

A specific preparation method of an iron-based stent is as follows: A nitrided iron-based matrix of a specification of 30008 with a wall thickness of 50 microns and an inner diameter of 1.45 mm was galvanized using a chemical plating method to obtain a zinc-containing protective layer that covered an entire surface of the iron-based matrix and had a uniform thickness (0.5 microns). The grain size of zinc in the zinc-containing protective layer was submicron. Later, an ethyl acetate solution of PDLLA with a weight-average molecular weight of 200 kDa was spray-coated, and after a solvent was dried, a corrosion-promoting layer that completely covered the zinc-containing protective layer was obtained. In the spray-coating process, one PTFE mandrel was added inside the iron-based matrix to obtain the corrosion-promoting layer with different thicknesses at inner and outer walls. A portion of the corrosion-promoting layer that was located at an outer wall of the iron-based matrix had a thickness of 15 microns, and a portion located at an inner wall had a thickness of 3 microns. An inner wall thickness ratio was 0.17, which was greater than an outer wall thickness ratio of 0.03. PDLLA, with a weight-average molecular weight of 30 kDa, was selected as a drug-loading polymer of the drug controlled-release layer; the ethyl acetate mixed solution of the PDLLA and sirolimus was spray-coated on a surface of the corrosion-promoting layer; in the spray-coating process, one PTFE mandrel was added inside the matrix, which completely blocked away the sprayed solution to prevent the solution from being deposited on the inner wall of the matrix; and after drying, the drug controlled-release layer that only covered the outer wall and a side wall of the matrix was obtained. A portion of the drug controlled-release layer that was located at the outer wall had a thickness of 2 microns, and the stent had a drug surface density of 1.4 μg/mm². The ratio of the molecular weight of the polymer in the corrosion-promoting layer to the molecular weight of the polymer in the drug controlled-release layer was 6.7.

The above stent was soaked in simulated body fluid; the whole stent was placed on a 37° C. constant-temperature air bath shaker; the simulated body fluid was replaced with fresh simulated body fluid every 7 days; after being soaked for 1, 7, 14, 28, and 60 days, the stent was taken out to test a drug release percentage and an iron-based matrix mass loss rate. The drug release percentages of the stent at various time points were 16.3%, 24.0%, 31.2%, 42.5% and 57.6% respectively. The mass loss rates of the stent at various time points were 3.45%, 10.67%, 29.33%, 44.34% and 67.98% respectively, and the corrosion of the iron-based matrix was initiated from an outer surface of the stent.

### Embodiment 11

A specific preparation method of an iron-based stent is as follows: A nitrided iron-based matrix of a specification of 30008 with a wall thickness of 50 microns and an inner diameter of 1.45 mm was galvanized using a chemical plating method to obtain a zinc-containing protective layer that covered an entire surface of the iron-based matrix and had a uniform thickness (0.5 microns). The grain size of zinc in the zinc-containing protective layer was submicron. Later, an ethyl acetate solution of PDLLA with a weight-average molecular weight of 200 kDa was spray-coated, and after a solvent was dried, a corrosion-promoting layer that completely covered the zinc-containing protective layer was obtained. In the spray-coating process, one PTFE mandrel was added inside the iron-based matrix to obtain the corrosion-promoting layer with different thicknesses at the inner and outer walls. A portion of the corrosion-promoting layer that was located at an outer wall of the iron-based matrix had a thickness of 4 microns, and a portion located at an inner wall had a thickness of 3 microns. An inner wall thickness ratio was 0.17, which was greater than an outer wall thickness ratio of 0.13. PDLLA, with a weight-average molecular weight of 30 kDa, was selected as a drug-loading polymer of the drug controlled-release layer; the ethyl acetate mixed solution of the PDLLA and sirolimus was spray-coated on a surface of the corrosion-promoting layer; in the spray-coating process, one PTFE mandrel was added inside the matrix, which completely blocked away the sprayed solution to prevent the solution from being deposited on the inner wall of the matrix; and after drying, the drug controlled-release layer that only covered the outer wall and a side wall of the matrix was obtained. A portion of the drug controlled-release layer that was located at the outer wall had a thickness of 2 microns, and the stent had a drug surface density of 1.4 μg/mm². The ratio of the molecular weight of the polymer in the corrosion-promoting layer to the molecular weight of the polymer in the drug controlled-release layer was 6.7.

The above stent was soaked in simulated body fluid; the whole stent was placed on a 37° C. constant-temperature air bath shaker; the simulated body fluid was replaced with fresh simulated body fluid every 7 days; after being soaked for 1, 7, 14, 28, and 60 days, the stent was taken out to test a drug release percentage and an iron-based matrix mass loss rate. The drug release percentages of the stent at various time points were 16.1%, 23.4%, 29.7%, 41.2% and 49.8% respectively. The mass loss rates of the stent at various time points were 1.54%, 9.87%, 19.98%, 33.45% and 48.7% respectively, and the corrosion of the iron-based matrix was initiated from an outer surface of the stent.

### Comparative Example 1

A specific preparation method of an iron-based stent is as follows: A nitrided iron-based matrix of a specification of 30008 with a wall thickness of 50 microns and an inner diameter of 1.45 mm was galvanized using an electroplating method to obtain a zinc-containing protective layer that covered an entire surface of the iron-based matrix and had a uniform thickness (1 micron). The grain size of zinc in the zinc-containing protective layer was submicron. Later, an ethyl acetate solution of PDLLA with a weight-average molecular weight of 80 kDa was spray-coated, and after a solvent was dried, a corrosion-promoting layer that completely covered the zinc-containing protective layer was obtained. In the spray-coating process, one PTFE mandrel was added inside the iron-based matrix to obtain the corrosion-promoting layer with different thicknesses at the inner and outer walls. A portion of the corrosion-promoting layer that was located at an outer wall of the iron-based matrix had a thickness of 10 microns, and a portion located at an inner wall had a thickness of 8 microns. An inner wall thickness ratio was 0.13, which was greater than an outer wall thickness ratio of 0.10. PDLLA, with a weight-average molecular weight of 5 kDa, was selected as a drug-loading polymer of the drug controlled-release layer; the ethyl acetate mixed solution of the PDLLA and sirolimus was spray-coated on a surface of the corrosion-promoting layer; in the spray-coating process, one PTFE mandrel was added inside the matrix, which completely blocked away the sprayed solution to prevent the solution from being deposited on the inner wall of the matrix; and after drying, the drug controlled-release layer that only covered the outer wall and a side wall of the matrix was obtained. A portion of the drug controlled-release layer that was located at the outer wall had a thickness of 2 microns, and the stent had a drug surface density of 1.4 μg/mm². The ratio of the molecular weight of the polymer in the corrosion-promoting layer to the molecular weight of the polymer in the drug controlled-release layer was 16.0.

The above stent was soaked in simulated body fluid; the whole stent was placed on a 37° C. constant-temperature air bath shaker; the simulated body fluid was replaced with fresh simulated body fluid every 7 days; after being soaked for 1, 7, 14, 28, and 60 days, the stent was taken out to test a drug release percentage and an iron-based matrix mass loss rate. The drug release percentages of the stent at various time points were 11.51%, 32.12%, 59.80%, 87.66% and 96.50% respectively. The mass loss rates of the stent at various time points were 18.92%, 34.57%, 68.31%, 89.52% and 93.21% respectively, and the corrosion of the iron-based matrix was initiated from an outer surface of the stent.

The above stent was implanted into the iliac arteries of rabbits; the rabbits were killed after different raising times; and the implanted vessel segments were taken out for analysis. The drug release percentage after 1 day of implantation of the stent was 25.3%; the drug release percentage after 30 days was 87.9%; the drug release percentage after 60 days was 98.4%; and no drugs were released in the further time. It was observed that the iron-based matrix started to be corroded after 10 days of implantation of the stent; the mass loss rate after 30 days of implantation was 47.9%; the mass loss rate after 180 days of implantation was 60.5%; and the iron-based stent was fully corroded (the mass loss rate was greater than 95%) after 2.5 years of implantation. After 14 days of implantation, there was no obvious endothelial overlying on the stent; after 30 days of implantation, there were partial endothelial overlying on the stent, obvious thrombosis on some stent bars, and abnormal colors of tissues around the stent bars; and after 60 days of implantation of the stent, the vascular restenosis was serious, and the lumen area was lost over 75%.

### Comparative Example 2

A specific preparation method of an iron-based stent is as follows: A nitrided iron-based matrix of a specification of 30008 with a wall thickness of 50 microns and an inner diameter of 1.45 mm was galvanized using an electroplating method to obtain a zinc-containing protective layer that covered an entire surface of the iron-based matrix and had a uniform thickness (1 micron). The grain size of zinc in the zinc-containing protective layer was submicron. Later, an ethyl acetate solution of PDLLA with a weight-average molecular weight of 200 kDa was spray-coated, and after a solvent was dried, a corrosion-promoting layer that completely covered the zinc-containing protective layer was obtained. In the spray-coating process, one PTFE mandrel was added inside the iron-based matrix to obtain the corrosion-promoting layer with different thicknesses at the inner and outer walls. A portion of the corrosion-promoting layer that was located at an outer wall of the iron-based matrix had a thickness of 10 microns, and a portion located at an inner wall had a thickness of 8 microns. An inner wall thickness ratio was 0.13, which was greater than an outer wall thickness ratio of 0.10. PDLLA, with a weight-average molecular weight of 200 kDa, was selected as a drug-loading polymer of the drug controlled-release layer; the ethyl acetate mixed solution of the PDLLA and sirolimus was spray-coated on a surface of the corrosion-promoting layer; in the spray-coating process, one PTFE mandrel was added inside the matrix, which completely blocked away the sprayed solution to prevent the solution from being deposited on the inner wall of the matrix; and after drying, the drug controlled-release layer that only covered the outer wall and a side wall of the matrix was obtained. A portion of the drug controlled-release layer that was located at the outer wall had a thickness of 1.6 microns, and the stent had a drug surface density of 1.4 μg/mm². The ratio of the molecular weight of the polymer in the corrosion-promoting layer to the molecular weight of the polymer in the drug controlled-release layer was 1.0.

The above stent was soaked in simulated body fluid; the whole stent was placed on a 37° C. constant-temperature air bath shaker; the simulated body fluid was replaced with fresh simulated body fluid every 7 days; after being soaked for 1, 7, 14, 28, and 60 days, the stent was taken out to test a drug release percentage and an iron-based matrix mass loss rate. The drug release percentages of the stent at various time points were 54.17%, 55.72%, 57.23%, 63.46% and 70.01% respectively. The mass loss rates of the stent at various time points were 0.62%, 0.77%, 3.95%, 7.70% and 35.17% respectively, and the corrosion of the iron-based matrix was initiated from an outer surface of the stent.

The SEM graphs of the iron-based stent of Embodiment 1 and the iron-based stent of Comparative example 2 are respectively as shown in (a) and (b) of FIG. 2; the coating on the surface of the iron-based stent of Embodiment 1 was smooth; and there were obvious protruding points formed by drug accumulation in the coating on the surface of the iron-based stent of Comparative example 2, indicating that drug accumulation was obvious if a degradable polymer with a large molecular weight was used to carry drugs, so that it was difficult to achieve a good drug controlled-release effect.

### Comparative Example 3

A specific preparation method of an iron-based stent is as follows: A nitrided iron-based matrix of a specification of 30008 with a wall thickness of 50 microns and an inner diameter of 1.45 mm was galvanized using an electroplating method to obtain a zinc-containing protective layer that covered an entire surface of the iron-based matrix and had a uniform thickness (1 micron). The grain size of zinc in the zinc-containing protective layer was submicron. Later, the sample was soaked in an ethyl acetate solution of PDLLA with a weight-average molecular weight of 200 kDa, and a corrosion-promoting layer that completely covered the zinc-containing protective layer was obtained by dip coating. A portion of the corrosion-promoting layer that was located at an outer wall of the iron-based matrix had a thickness of 10 microns, and a portion located at an inner wall had a thickness of 10 microns. An inner wall thickness ratio was 0.10, which was greater than an outer wall thickness ratio of 0.10. PDLLA, with a weight average molecular weight of 30 kDa, was selected as a drug carrying polymer of the drug controlled-release layer; an ethyl acetate mixed solution of PDLLA and sirolimus was attached to a surface of the corrosion-promoting layer by ink-jet printing; and after drying, a drug controlled-release layer that only covered the outer wall of the matrix was obtained. A portion of the drug controlled-release layer that was located at the outer wall had a thickness of 5 microns, and the stent had a drug surface density of 1.4 μg/mm². The ratio of the molecular weight of the polymer in the corrosion-promoting layer to the molecular weight of the polymer in the drug controlled-release layer was 6.7.

The above stent was soaked in simulated body fluid; the whole stent was placed on a 37° C. constant-temperature air bath shaker; the simulated body fluid was replaced with fresh simulated body fluid every 7 days; after being soaked for 1, 7, 14, 28, and 60 days, the stent was taken out to test a drug release percentage and an iron-based matrix mass loss rate. The drug release percentages of the stent at various time points were 10.1%, 20.9%, 21.4%, 32.5% and 51.2% respectively. The mass loss rates of the stent at various time points were 0.34%, 1.00%, 4.21%, 7.99% and 43.25% respectively.

### Comparative Example 4

A specific preparation method of an iron-based stent is as follows: A nitrided iron-based matrix of a specification of 30008 with a wall thickness of 50 microns and an inner diameter of 1.45 mm was galvanized using a vapor deposition method to obtain a zinc-containing protective layer that covered an entire surface of the iron-based matrix and had a uniform thickness (1 micron). The grain size of zinc in the zinc-containing protective layer was submicron. Later, an ethyl acetate solution of PDLLA with a weight-average molecular weight of 200 kDa was ink-jet printed, and after a solvent was dried, a corrosion-promoting layer that completely covered the zinc-containing protective layer was obtained. In the spray-coating process, one PTFE mandrel was added inside the iron-based matrix to obtain the corrosion-promoting layer with different thicknesses at the inner and outer walls. A portion of the corrosion-promoting layer that was located at an outer wall of the iron-based matrix had a thickness of 8 microns, and a portion located at an inner wall had a thickness of 10 microns. An inner wall thickness ratio was 0.10, which was greater than an outer wall thickness ratio of 0.13. PDLLA, with a weight-average molecular weight of 30 kDa, was selected as a drug-loading polymer of the drug controlled-release layer; the ethyl acetate mixed solution of the PDLLA and sirolimus was spray-coated on a surface of the corrosion-promoting layer; in the spray-coating process, one PTFE mandrel was added inside the matrix, which completely blocked away the sprayed solution to prevent the solution from being deposited on the inner wall of the matrix; and after drying, the drug controlled-release layer that only covered the outer wall and a side wall of the matrix was obtained. A portion of the drug controlled-release layer that was located at the outer wall had a thickness of 2 microns, and the stent had a drug surface density of 1.4 μg/mm². The ratio of the molecular weight of the polymer in the corrosion-promoting layer to the molecular weight of the polymer in the drug controlled-release layer was 6.7.

The above stent was soaked in simulated body fluid; the whole stent was placed on a 37° C. constant-temperature air bath shaker; the simulated body fluid was replaced with fresh simulated body fluid every 7 days; after being soaked for 1, 7, 14, 28, and 60 days, the stent was taken out to test a drug release percentage and an iron-based matrix mass loss rate. The drug release percentages of the stent at various time points were 9.9%, 20.5%, 21.9%, 32.5% and 48.7% respectively. The mass loss rates of the stent at various time points were 0.41%, 1.10%, 1.92%, 6.92% and 48.45% respectively, and the corrosion of the iron-based matrix was initiated from an inner surface of the stent.

### Comparative Example 5

A specific preparation method of an iron-based stent is as follows: A nitrided iron-based matrix of a specification of 30008 with a wall thickness of 50 microns and an inner diameter of 1.45 mm was galvanized using an electroplating method to obtain a zinc-containing protective layer that covered an entire surface of the iron-based matrix and had a uniform thickness (1 micron). The grain size of zinc in the zinc-containing protective layer was submicron. Later, an ethyl acetate solution of PDLLA, with a weight-average molecular weight of 200 kDa, was spray-coated, and after a solvent was dried, a corrosion-promoting layer that completely covered the zinc-containing protective layer was obtained. In the spray-coating process, one PTFE mandrel was added inside the iron-based matrix to obtain the corrosion-promoting layer with different thicknesses at inner and outer walls. A portion of the corrosion-promoting layer that was located at an outer wall of the iron-based matrix had a thickness of 10 microns, and a portion located at an inner wall had a thickness of 8 microns. An inner wall thickness ratio was 0.13, which was greater than an outer wall thickness ratio of 0.10.

The above stent was soaked in simulated body fluid; the whole stent was placed on a 37° C. constant-temperature air bath shaker; the simulated body fluid was replaced with fresh simulated body fluid every 7 days; after being soaked for 1, 7, 14, 28, and 60 days, the stent was taken out to test mass loss rates of the iron-based matrix. The mass loss rates of the stent at various time points were 0.2%, 0.84%, 2.07%, 7.56% and 38.54% respectively, and the corrosion of the iron-based matrix was initiated from an outer surface of the stent.

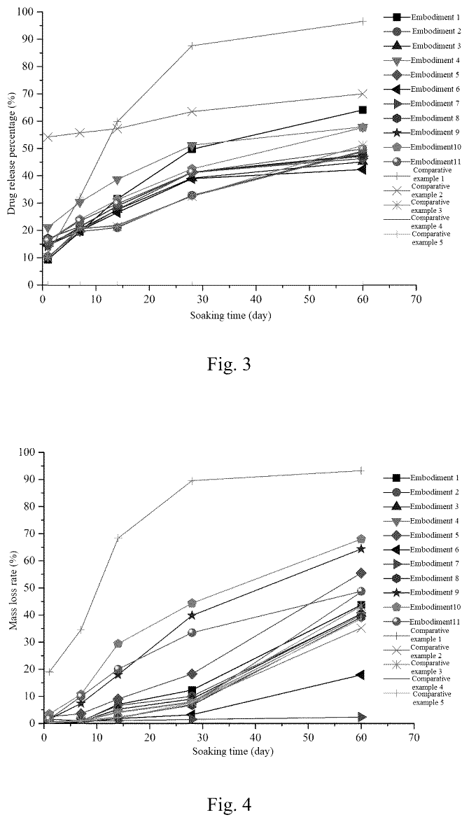

The drug release and corrosion behaviors of the stents of Embodiments 1-11 and Comparative examples 1-5 in the simulated body fluid are compared as a whole in FIG. 3 and FIG. 4. The types of the polymers, the molecular weights of the polymers, the thickness of the zinc-containing protective layer, the thickness of the corrosion-promoting layer, and the dosage matching and spatial layout of all the above items affect the corrosion behavior and the drug release behavior of the iron-based matrix.

The time points for initiating the corrosion of the absorbable iron-based stent of the present disclosure are suitable, and the corrosion of the iron-based matrix is initiated from the outer surface of the stent. According to the corrosion rule in vitro, it is determined that after implantation, the time point when the corrosion is initiated is about 3 to 6 months after implantation. The drug release rule is also reasonable. There is no serious drug release, and the drug is continuously and slowly released. According to the drug release curves in vitro, it is determined that after implantation, the action period of drug release is about 2 to 6 months after implantation, and the drug is continuously and uniformly released at each stage. In contrast, for the iron-based stents of Comparative examples 1 to 5, their drug release and corrosion behaviors deviate far from an ideal situation, and the stents are less safe and effective. For example, in the comparative examples, if the molecular weight of the polymer in the corrosion-promoting layer is too small, the iron-based matrix will start to be corroded prematurely; and if the molecular weight of the polymer in the corrosion-promoting layer is too large, it is difficult to prepare the corrosion-promoting layer, and the stent starts to be corroded too late, both of which are not consistent with the requirements of a tissue repair process. For another example, if the molecular weight of the polymer in the drug controlled-release layer is too small, the polymer will be degraded too quickly, so that the overall drug release is fast, and the action period of the drug is too short. If the molecular weight of the polymer in the drug controlled-release layer is too large, there will be serious drug release in the initial stage of implantation, the drug release will be weak in the later stage, and the utilization rate of the drug is low.

The technical features of the embodiments described above can be arbitrarily combined. In order to make the description concise, all possible combinations of various technical features in the above embodiments are not completely described. However, the combinations of these technical features should be considered as the scope described in the present specification as long as there is no contradiction in them.

The above-mentioned embodiments only express several implementation modes of the present invention, and their descriptions are more specific and detailed, but they cannot be understood as limiting the patent scope of the present invention. It should be noted that those of ordinary skill in the art can further make various transformations and improvements without departing from the concept of the present invention, and these transformations and improvements all fall within the protection scope of the present invention. Therefore, the protection scope of the patent of the present invention shall be subject to the appended claims.

1. An absorbable iron-based instrument, comprising an iron-based matrix, a zinc-containing protective layer, a corrosion-promoting layer and a drug controlled-release layer, wherein the iron-based matrix is provided with an outer wall, an inner wall and a side wall; the zinc-containing protective layer covers at least the outer wall and the inner wall of the iron-based matrix; the corrosion-promoting layer fully covers the zinc-containing protective layer; the drug controlled-release layer partially covers at least the corrosion-promoting layer; the corrosion-promoting layer and the drug controlled-release layer each contain degradable polymers; the weight-average molecular weight of the degradable polymer in the corrosion-promoting layer is greater than the weight-average molecular weight of the degradable polymer in the drug controlled-release layer; and the thickness ratio of a portion of the zinc-containing protective layer that is located at the inner wall to a portion of the corrosion-promoting layer that is located at the inner wall is greater than the thickness ratio of a portion of the zinc-containing protective layer that is located at the outer wall to a portion of the corrosion-promoting layer that is located at the outer wall.

2. The absorbable iron-based instrument according to claim 1, wherein the weight-average molecular weight of the degradable polymer in the corrosion-promoting layer is at least twice as large as the weight-average molecular weight of the degradable polymer in the drug controlled-release layer.

3. The absorbable iron-based instrument according to claim 1, wherein the weight-average molecular weight of the degradable polymer in the corrosion-promoting layer is 100-1000 kDa, and the weight-average molecular weight of the degradable polymer in the drug controlled-release layer is 8-50 kDa.

4. The absorbable iron-based instrument according to claim 1, wherein the thickness ratio of the portion of the zinc-containing protective layer that is located at the inner wall to the portion of the corrosion-promoting layer that is located at the inner wall is 0.05-0.7, and the thickness ratio of the portion of the zinc-containing protective layer that is located at the outer wall to the portion of the corrosion-promoting layer that is located at the outer wall is 0.03-0.5.

5. The absorbable iron-based instrument according to claim 1, wherein the thickness of the portion of the zinc-containing protective layer that is located at the outer wall is 0.5-2.0 microns, and the thickness of the portion of the zinc-containing protective layer that is located at the inner wall is 0.5-2.0 microns.

6. The absorbable iron-based instrument according to claim 1, wherein the portion of the corrosion-promoting layer that is located at the outer wall has a thickness range from 4 to 15 microns, and the portion of the corrosion-promoting layer that is located at the inner wall has a thickness range from 3 to 10 microns.

7. The absorbable iron-based instrument according to claim 1, wherein the thickness of the drug controlled-release layer is less than or equal to 5 microns.

8. The absorbable iron-based instrument according to claim 1, wherein the material of the zinc-containing protective layer is pure zinc or a zinc alloy, and grain sizes of the pure zinc and the zinc alloy are both submicron.

9. The absorbable iron-based instrument according to claim 1, wherein the zinc-containing protective layer covers the outer wall, the inner wall and the side wall of the iron-based matrix; the corrosion-promoting layer covers the entire surface of the zinc-containing protective layer; and the drug controlled-release layer covers at least the portion of the corrosion-promoting layer that is located at the outer wall.

10. The absorbable iron-based instrument according to claim 1, wherein there is at least one corrosion-promoting layer; and when there is a plurality of corrosion-promoting layers, the degradable polymers in the different corrosion-promoting layers have different molecular weights and/or are of different types.
